SEQUENCE LISTING (2 sequences)

SEQ ID NO: 1 - DNA, 477 bp, Homo sapiens
  source 1..477: mol_type = genomic DNA
tctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtag

SEQ ID NO: 2 - protein, 158 aa, Homo sapiens
SVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

(19) United States
(12) Patent Application Publication
(10) Pub. No.: US 2026/0103495 A1
(43) Pub. Date: Apr. 16, 2026

(54) PERSONALIZED MUC1-C INDUCED PLURIPOTENT STEM CELL AND DENDRITIC CELL BASED VACCINES

(71) Applicant: Dana-Farber Cancer Institute, Inc., Boston, MA (US)

(72) Inventor: Donald Kufe, Boston, MA (US)

(73) Assignee: Dana-Farber Cancer Institute, Inc., Boston, MA (US)

(21) Appl. No.: 19/116,785

(22) PCT Filed: Sep. 29, 2023
(86) PCT No.: PCT/US2023/075603

§ 371 Date: Mar. 28, 2025

Related U.S. Application Data
(60) Provisional application No. 63/377,956, filed on Sep. 30, 2022.

(51) Int. Cl. C07K 14/47; A61K 39/00; A61P 37/04; C12N 5/074; C12N 5/0784; C12N 5/16

(52) U.S. Cl. CPC C07K 14/4727; A61K 39/0011; A61P 37/04; C12N 5/0639; C12N 5/0696; C12N 5/16; A61K 2039/5154; C12N 2506/1307; C12N 2510/00

(57) ABSTRACT

The present disclosure relates to engineered cells expressing heterologous MUC1, MUC1-C or MUC1-C cytoplasmic domain for generating induced pluripotent stem cells that are autologous to a subject.

### STATEMENT REGARDING SEQUENCE LISTING

This application claims priority to U.S. Provisional Application No. 63/377,956 filed on Sep. 30, 2022 and which is incorporated herein in its entirety by reference.

The Sequence Listing associated with this application is provided in XML format in lieu of paper copy, and is hereby incorporated by reference into the specification. The name of the XML file containing the Sequence Listing is “91016-364218 Sequence Listing_ST26.” The XML file is 3 KB, was created on Aug. 9, 2022, and is being submitted electronically, concurrent with the filing of this specification.

### FIELD

The present disclosure relates to novel engineered cells expressing nucleic acid sequences encoding MUC1 protein or fragments thereof, including the MUC1-C subunit or the MUC1-C cytoplasmic domain, and methods of generating induced pluripotent stem cells that are autologous to a patient for use in immunizing against cancer.

### BACKGROUND OF THE INVENTION

Blood and bone marrow cancers, such as leukemia, lymphoma, and myeloma make up almost 10% of new cancer cases that will be diagnosed in the U.S. in 2022, with solid tumors making up 90% of adult cancers. Cancer vaccines offer an immuno-therapeutic strategy in which cancer antigens may be used to overcome immune suppression and induce immunity but may not be personalized to an individual. Personalized vaccines that do exist require sampling the individual's cancer. Thus, limitations to personalized cancer vaccines remain.

### SUMMARY OF THE INVENTION

In one embodiment described herein is a fibroblast comprising a heterologous nucleic acid sequence encoding MUC1-C operably linked to at least one regulatory sequence, wherein expression of MUC1-C is in an amount that induces pluripotency. In one aspect, the nucleic acid sequence encodes a polypeptide comprising the amino acid sequence of SEQ ID NO: 2 or a functional variant thereof. In another aspect, the MUC1-C comprises a MUC1-C cytoplasmic domain. In another aspect, the fibroblast is autologous to a patient. In another aspect, the fibroblast comprises a dermal fibroblast, a lung fibroblast, or an intestinal fibroblast. In another aspect, the fibroblast further comprises a nucleic acid sequence encoding for at least one of OCT3/4, SOX2, KLF4, and C-MYC, wherein the at least one of OCT3/4, SOX2, KLF4, and C-MYC consists of an endogenous nucleic acid.

In another embodiment described herein is a fibroblast comprising a pluripotency activator consisting of one or more of MUC1, MUC1-C, or MUC1-C cytoplasmic domain. In one aspect, the pluripotency activator is not OCT3/4, SOX2, KLF4, and C-MYC or combinations thereof.

In another embodiment described herein is a fibroblast comprising oligonucleotide means for inducing pluripotency, operably linked to at least one regulatory sequence, wherein expression of the oligonucleotide means for inducing pluripotency is in an amount that induces pluripotency. In another aspect, the oligonucleotide means for inducing pluripotency comprises the nucleic acid sequence of SEQ ID NO: 1, or a functional variant thereof. In another aspect, the oligonucleotide means for inducing pluripotency encodes a polypeptide comprising the amino acid sequence of SEQ ID NO: 2, or a functional variant thereof.

Another aspect described herein is a method of reprogramming a fibroblast to induce pluripotency comprising: (a) expressing the MUC1-C in the fibroblast as described herein; (b) inducing pluripotency of the fibroblast; and (c) generating an induced pluripotent stem cell from the fibroblast. In another aspect, the induced pluripotent stem cell is undifferentiated. In another aspect, the induced pluripotent cell is autologous to the fibroblast.

Another aspect described herein is a hybrid cell comprising: (a) an autologous dendritic cell; and (b) the induced pluripotent stem cell as described herein, wherein the dendritic cell and induced pluripotent stem cell are fused. In another aspect, the induced pluripotent cell comprises an expression signature. In another aspect, the expression signature comprises an expression signature substantially similar expression to the expression signature of cancer stem cells. In another aspect, the expression signature encodes proteins from which at least one peptide is derived.

Another aspect described herein is a pharmaceutical composition comprising the hybrid cell of as described herein.

Another aspect described herein is a method of inducing an immune response in a subject in need thereof comprising administering to the subject an effective amount of any of the pharmaceutical compositions described herein.

Another aspect described herein is a method of treating cancer in a subject in need thereof comprising administering to the subject an effective amount of any of the pharmaceutical compositions described herein.

In one aspect, the method further comprises presenting the peptide. In another aspect, the method further comprises inducing an immune response. In another aspect, the method further comprises irradiating the hybrid cell prior to administration to the patient.

In one aspect, the cancer comprises a solid tumor or liquid cancer.

Another aspect described herein is a method of making a personalized cancer vaccine comprising: (a) obtaining a fibroblast from a patient; (b) introducing in the fibroblast, an exogenous nucleic acid sequence encoding a pluripotency activator gene operably linked to at least one regulatory sequence; (c) expressing the pluripotency activator in the fibroblast; (d) inducing pluripotency of the fibroblast; (e) generating an autologous induced pluripotent stem cell from the fibroblast; (f) fusing the autologous induced pluripotent stem cell with an autologous dendritic cell to generate a hybrid cell; and (g) formulating the hybrid cell into a pharmaceutical composition. In one aspect, the induced pluripotent stem cell comprises an expression signature substantially similar to the expression signature of cancer stem cells. In another aspect, the expression signature encodes proteins from which at least one peptide is derived. In another aspect, the hybrid cell presents the peptide.

### BRIEF DESCRIPTION OF THE FIGURES

FIG. 1A. shows example lysates from human MRC5 fibroblasts transduced to express a control empty vector or one encoding MUC1-C that were immunoblotted with antibodies against the indicated proteins. FIG. 1B. shows example MRC5 fibroblasts expressing the empty vector or MUC1-C seeded in ultra-low culture plates in medium used for iPSCs. Sphere formation is shown with representative examples in duplicate wells.

FIG. 2 shows exemplary images of MUC1-C inducing (i) NOTCH1 and pluripotency factor expression and (ii) self-renewal capacity in human dermal fibroblasts (HDFs). FIG. 2A shows lysates from HDFs transduced to express a control empty vector or one encoding MUC1-C that were immunoblotted with antibodies against the indicated proteins. FIG. 2B shows an exemplary image of HDFs expressing an empty vector or MUC1-C, in which the HDFs were seeded in ultra-low culture plates in medium used for iPSCs. Sphere formation is shown in the representative examples.

FIG. 3 shows a volcano plot generated from RNA-seq analysis of HDFs without and with MUC1-C expression in which MUC1-C down-regulated genes are depicted on the left and up-regulated genes on the right.

FIG. 4 shows numbers of MUC1-C-induced down-regulated and up-regulated genes using the indicated Log2 Fold Change cutoffs.

FIG. 5 shows analysis of the RNA-seq data using a Gene Ontology gene signature set, which demonstrates that MUC1-C (i) up-regulates the interferon type I/II responses and other inflammatory associated pathways, and (ii) down-regulates specific pathways associated with cell cycle progression.

FIG. 6 shows Gene Set Enrichment Analysis (GSEA) of the RNA-seq data using the cell cycle G2/M phase and cell cycle checkpoint gene signatures down regulated by MUC1-C expression.

FIG. 7 shows GSEA of the RNA-seq data using the DNA regulation and response to interferon gamma gene signatures.

FIG. 8 shows GSEA of the RNA-seq data using the indicated Reactome gene signatures.

FIG. 9 shows network analysis of the RNA-seq data for putative transcription factor binding, which indicates that MUC1-C up-regulates transcription factors associated with inflammatory pathways and down-regulates those associated with cell cycle progression.

FIG. 10 shows GSEA analysis of the RNA-seq data using the indicated KEGG pathways

FIG. 11 shows GSEA of the RNA-seq data using the indicated HALLMARK genes signatures that are up-regulated (left) and down-regulated (right) by MUC1-C expression.

FIG. 12 shows GSEA of the RNA-seq data using the other indicated curated gene sets in the Molecular Signature Database (MSigDB).

FIG. 13 depicts a model based on the findings that MUC1-C induces upregulation of inflammatory genes and downregulation of cell cycle genes in HDFs in association with increases in the expression of drug transporters and effectors of the epithelial-mesenchymal transition (EMT). These findings reflect the effects of MUC1-C in driving the CSC state and extend to performing analyses of teratoma formation in immunodeficient mice, lineage plasticity and treatment resistance.

FIG. 14 is a schematic outlining the steps of MUC1-C overexpression in normal skin fibroblasts to induce iPSCs for the generation of dendritic cell-based personalized fusion vaccines.

### DETAILED DESCRIPTION OF THE INVENTION

The present disclosure relates to engineered cells expressing nucleic acid sequences encoding MUC1 protein, the MUC1-C subunit or the MUC1-C cytoplasmic domain. In some embodiments, the disclosed engineered cells can be used to generate induced pluripotent stem cells that are personal to or autologous to a patient or subject, such as for use in immunizing a patient or subject against cancer.

Unless otherwise defined, all technical and scientific terms used herein have the same meaning as commonly understood by one of ordinary skill in the art. In case of conflict, the present document, including definitions, will control. Methods and materials are described below, although methods and materials similar or equivalent to those described herein may be used in practice or testing of the present disclosure. All publications, patent applications, patents and other references mentioned herein are incorporated by reference in their entirety. The materials, methods, and examples disclosed herein are illustrative only and not intended to be limiting.

As used herein, the articles “a,” “an,” and “the” are used herein to refer to one or to more than one (i.e., to at least one) of the grammatical object of the article. By way of example, “an element” can mean one element or more than one element.

In this application, the use of “or” means “and/or” unless stated otherwise. The terms “and/or” and “any combination thereof” and their grammatical equivalents as used herein, may be used interchangeably. These terms may convey that any combination is specifically contemplated. Solely for illustrative purposes, the following phrases “A, B, and/or C” or “A, B, C, or any combination thereof” may mean “A individually; B individually; C individually; A and B; B and C; A and C; and A, B, and C.” The term “or” may be used conjunctively or disjunctively, unless the context specifically refers to a disjunctive use.

Furthermore, use of the term “including” as well as other forms, such as “include,” “includes,” and “included,” is not limiting.

Reference in the specification to “some embodiments,” “an embodiment,” “one embodiment” or “other embodiments” or “some aspects”, “an aspect” or “one aspect” means that a particular feature, structure, or characteristic described in connection with the embodiment and/or aspect is included in at least some embodiments and/or aspects, but not necessarily all, of the present disclosure.

As used in this specification and the claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification may be implemented with respect to any method or composition of the disclosure, and vice versa. Furthermore, compositions of the present disclosure may be used to achieve methods of the present disclosure.

As used herein, the term “about” or “approximately” refers to a quantity, level, value, number, frequency, percentage, dimension, size, amount, weight or length that varies by as much as 10, 9, 8, 7, 6, 5, 4, 3, 2 or 1% to a reference quantity, level, value, number, frequency, percentage, dimension, size, amount, weight or length. In some embodiments, the terms “about” or “approximately” when preceding a numerical value indicates the value plus or minus a range of 10%, 5%, or 1%.

As used herein, “an effective amount” refers to an amount that causes relief of symptoms of a disorder or disease as noted through clinical testing and evaluation, patient observation, and/or the like. An “effective amount” may further designate a dose that causes a detectable change in biological or chemical activity. The detectable changes may be further quantified by one skilled in the art for the relevant mechanism or process. Moreover, an “effective amount” may designate an amount that maintains a desired physiological state, i.e., reduces or prevents significant decline and/or promotes improvement in the condition of interest. An “effective amount” may further refer to a “therapeutically effective amount”. In some embodiments, expression of “an effective amount” refers to an amount of expression of MUC1, MUC1-C, MUC1-C cytoplasmic domain, or fragments thereof to induce pluripotency in fibroblasts.

The term “expression signature” as used herein refers to the profile of specific genes expressed in pluripotent stem cells that may be similar, substantially similar or identical to cancer cells and cancer stem cells (CSCs). Exemplary expression signatures may include individual genes or combinations of genes. For example, in glia and lung CSCs, expression signatures of upregulated and downregulated genes may include, but are not limited to: CDC6, MCM2-7, MSH2, CD44, CD24, TGFβ1, FN1. In breast cancer CSCs, expression signatures of upregulated and downregulated genes may include, but are not limited to: IGFBP-3, IGFBP-4, FBN1, MMP-2, PDGFRα, TWIST1, VIM, TGFβ1, FN1, CDH1, ERB-B3, KRT5, KRT8, KRT14, KRT18, MUC1, OCLN. In other cancer stem cells expression signatures of upregulating and downregulating genes may include, but are not limited to: APOE, ABCA1, NRIH3, PLTP, AGR2, BRAF, EGFR, KRA, MAP2K1, PIK3CA, KRAS, or PEA15. (Hsu et al., 2016). In some aspects, the expression signature of upregulated and downregulated genes may include, but are not limited to, MX1, JFIT2, OAS1, IF144, STAT1, BST2, MYC, NOTCH1, CDK4, CCND3, or CDC25A, which contribute to the inflammatory memory response and self-renewal capacity of CSCs.

“Induce an immune response” as used herein refers to the stimulation, generation and/or eliciting of an immune response in a mammal. An optimal immune response for a cancer vaccine is to prime the host's immune system to target antigens on pluripotent cells and provide immunity to cancer types that express the same proteins. This includes the generation of T cells, such as cytotoxic T cells; B cells; macrophages; and other immune effectors, as well as the generation of antibodies.

As used herein, “a pluripotent cell” or “pluripotent stem cell” is a cell that has the ability to differentiate into cells derived from all three embryonic germ layers —endoderm, mesoderm and ectoderm and thus all cell types. An “induced pluripotent stem cell” encompasses pluripotent stem cells that can be cultured over a period of time while maintaining the ability to differentiate into all cell types and are derived from differentiated somatic cells. “Induced pluripotent stem cells” are cells in which pluripotency is induced, for example, by the introduction of MUC1 genes and/or proteins, including the MUC1-C subunit or MUC1-C cytoplasmic domain, a fragment, or functional variants thereof, into a somatic cell that when expressed, induces the somatic cell into a less differentiated state, resulting in a pluripotent stem cell. Thus, pluripotent stem cells are considered to be partially differentiated or fully undifferentiated when they have not committed to a specific lineage.

As used herein, a “pluripotency activator” refers to a heterologous gene and/or their protein products, associated with inducing pluripotency. A pluripotency activator may also be considered as one that induces reprogramming of a somatic cell to an iPSC. Pluripotency activators may include, but are not limited to genes and proteins including, for example transcription factors and histones. Another example of a pluripotency activator is MUC1 or individual domains within MUC1 that induce pluripotency, including for example, the MUC1-C subunit, the MUC1-C cytoplasmic domain, a fragment, as wild-type sequences or functional variants thereof. Thus, the MUC1 genes, the proteins they express, and specific domains within the proteins may be considered “pluripotency activators” for inducing pluripotency. Thus, in some embodiments, the pluripotency activator is MUC1, MUC1-C subunit, the MUC1-C cytoplasmic domain, or functional variants thereof. In some embodiments the pluripotency factor comprises MUC1. In one aspect the pluripotency factor comprises MUC1-C. In another aspect the pluripotency activator comprises MUC1-C cytoplasmic domain. In some embodiments, the one or more pluripotency factors are expressed in a somatic cell, such as fibroblasts. For example, disclosed herein is expression of heterologous pluripotency activators MUC1, MUC1-C, the MUC1-C cytoplasmic domain, and/or fragments thereof in fibroblasts, inducing pluripotency in the fibroblasts and generating iPSCs.

Certain pluripotency activators can be excluded as well. In some embodiments, the one or more pluripotency activators expressed in somatic cells do not include one or more heterologous Yamanaka factors, such as heterologous Oct3/4, Sox2, Klf4, c-Myc, either individually or in any combination thereof, or any heterologous regulatory sequences thereof.

As used herein, “reprogramming” refers to any inducement of somatic cells to de-differentiate, i.e. movement of the differentiation status of a cell along the spectrum toward a less differentiated or completely undifferentiated state. The differentiation status of a cell is a spectrum, with a terminally differentiated state at one end, and a de-differentiated or undifferentiated state (pluripotent state) at the other end. Reprogramming includes moving the differentiation state of a somatic cell to a lesser differentiated state or to an undifferentiated state (pluripotent state).

“Polynucleotide” or “oligonucleotide” as used herein refers to a polymeric form of nucleotides or nucleic acid sequences of any length, either ribonucleotides or deoxyribonucleotides. This term refers only to the primary structure of the molecule. Thus, this term includes double and single stranded DNA, triplex DNA, as well as double and single stranded RNA. It also includes modified forms, for example, by methylation and/or by capping, and unmodified forms of the polynucleotide. The term is also meant to include molecules that include non-naturally occurring or synthetic nucleotides as well as nucleotide analogs.

The term “oligonucleotide means for inducing pluripotency” as used herein refers to nucleic acid sequence that encode for MUC1, MUC1-C or the MUC1-C cytoplasmic domain, a fragment or functional variant thereof, that induce pluripotency and statutory equivalents. In one aspect, the oligonucleotide means for inducing pluripotency comprises the nucleic acid sequence of MUC1, fragments, or functional variants thereof. In another aspect, the oligonucleotide means for inducing pluripotency comprises the nucleic acid sequence of MUC1-C, fragments, or functional variants thereof. In another aspect, the oligonucleotide means for inducing pluripotency comprises the nucleic acid sequence of MUC1-C extracellular domain, fragments, or functional variants thereof. In yet another aspect, the oligonucleotide means for inducing pluripotency comprises MUC1-C cytoplasmic domain, fragments, or functional variants thereof. In one aspect, the oligonucleotide means for inducing pluripotency comprises the nucleic acid sequence of SEQ ID NO: 1 or a functional variant thereof (e.g., a nucleic acid having at least about 80%, 85%, 90%, 95%, 96%, 97%, 98%, or 99% sequence identity with SEQ ID NO: 1), or a codon degenerate variant of SEQ ID NO: 1. In another aspect, the oligonucleotide means for inducing pluripotency comprises a nucleic acid encoding a polypeptide comprising the amino acid sequence of SEQ ID NO: 2 or a functional variant thereof (e.g., a nucleic acid having at least about 80%, 85%, 90%, 95%, 96%, 97%, 98%, or 99% sequence identity with SEQ ID NO: 2. Unless otherwise stated, nucleic acid sequences in the text of this specification are given, when read from left to right, in the 5′ to 3′ direction.

The terms “transfection,” “transformation,” “nucleofection,” or “transduction” as used herein refer to the introduction of one or more exogenous polynucleotides into a host cell or organism by using physical, chemical, and/or electrical methods. The nucleic acid sequences and vectors disclosed herein may be introduced into a cell or organism by any such methods, including, for example, by electroporation, calcium phosphate co-precipitation, strontium phosphate DNA co-precipitation, liposome mediated-transfection, DEAE dextran mediated-transfection, polycationic mediated-transfection, tungsten particle-facilitated microparticle bombardment, viral, and/or non-viral mediated transfection. In some cases, the method of introducing nucleic acids into the cell or organism involves the use of viral, retroviral, lentiviral, or transposon, or transposable element-mediated (e.g., Sleeping Beauty) vectors.

“Polypeptide”, “peptide”, and their grammatical equivalents as used herein refer to a polymer of amino acid residues. The polypeptide may optionally include glycosylation or other modifications typical for a given protein in a given cellular environment. Polypeptides and proteins disclosed herein (including functional portions and functional variants thereof) may comprise synthetic amino acids in place of one or more naturally-occurring amino acids.

The terms “identical” and its grammatical equivalents as used herein or “sequence identity” in the context of two nucleic acid sequences or amino acid sequences of polypeptides refer to the residues in the two sequences which are the same when aligned for maximum correspondence over a specified comparison window. A “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman, Adv. Appl. Math., 2:482 (1981); by the alignment algorithm of Needleman and Wunsch, J. Mol. Biol., 48:443 (1970); by the search for similarity method of Pearson and Lipman, Proc. Nat. Acad. Sci U.S.A., 85:2444 (1988); by computerized implementations of these algorithms (including, but not limited to CLUSTAL in the PC/Gene program by Intelligentics, Mountain View Calif., GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp, Gene, 73:237-244 (1988) and Higgins and Sharp, CABIOS, 5:151-153 (1989): Corpet et al., Nucleic Acids Res., 16:10881-10890 (1988); Huang et al., Computer Applications in the Biosciences, 8:155-165 (1992); and Pearson et al., Methods in Molecular Biology, 24:307-331 (1994). Alignment may also be performed by inspection and manual alignment. In one class of embodiments, the polypeptides herein are at least 80%, 85%, 90%, 98% 99% or 100% identical to a reference polypeptide, or a fragment thereof, e.g., as measured by BLASTP (or CLUSTAL, or any other available alignment software) using default parameters. Similarly, nucleic acids may also be described with reference to a starting nucleic acid, e.g., they may be 50%, 60%, 70%, 75%, 80%, 85%, 90%, 98%, 99% or 100% identical to a reference nucleic acid or a fragment thereof, e.g., as measured by BLASTN (or CLUSTAL, or any other available alignment software) using default parameters. When one molecule is said to have a certain percentage of sequence identity with a larger molecule, it means that when the two molecules are optimally aligned, the percentage of residues in the smaller molecule finds a match residue in the larger molecule in accordance with the order by which the two molecules are optimally aligned.

The term “substantially identical” and its grammatical equivalents as applied to nucleic acid or amino acid sequences mean that a nucleic acid or amino acid sequence comprises a sequence that has at least 95% sequence identity with a reference sequence using the programs described above, e.g., BLAST, using standard parameters. For example, the BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1992)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. In some embodiments, the substantial identity exists over a region of the sequences that is at least about 50 residues in length, over a region of at least about 100 residues, and in some embodiments, the sequences are substantially identical over at least about 150 residues. In some embodiments, the sequences are substantially identical over the entire length of the coding regions. “Substantially similar” may also refer to the expression signature of specific genes expressed in pluripotent stem cells that may be at least 50%, at least 60%, at least 70%, at least 80%, at least 90% or at least 95% identical to cancer cells and cancer stem cells. “Homology” is generally inferred from sequence identity between two or more nucleic acids or proteins (or sequences thereof). The precise percentage of identity between sequences that is useful in establishing homology varies with the nucleic acid and protein at issue, but as little as 25% sequence identity is routinely used to establish homology. Higher levels of sequence identity, e.g., 30%, 40%, 50%, 60%, 70%, 80%, 90%, 95%, or 99% or more may also be used to establish homology. Methods for determining sequence identity percentages (e.g., BLASTP and BLASTN using default parameters) are described herein and are generally available. Nucleic acids and/or nucleic acid sequences are “homologous” when they are derived, naturally or artificially, from a common ancestral nucleic acid or nucleic acid sequence. Proteins and/or protein sequences are “homologous” when their encoding DNAs are derived, naturally or artificially, from a common ancestral nucleic acid or nucleic acid sequence. The homologous molecules may be termed “homologs.” For example, any naturally occurring proteins may be modified by any available mutagenesis method. When expressed, this mutagenized nucleic acid encodes a polypeptide that is homologous to the protein encoded by the original nucleic acid.

Also contemplated and included herein are nucleic acid molecules that hybridize to the disclosed sequences. Hybridization conditions may be mild, moderate, or stringent, as is warranted.

As will be appreciated by the skilled practitioner, slight changes in nucleic acid sequence do not necessarily alter the amino acid sequence of the encoded polypeptide. This disclosure embraces the degeneracy of codon usage as would be understood by one of ordinary skill in the art. For example, as known in the art, different codons will code for the same amino acid.

As used herein, the phrase “codon degenerate variant” when used with reference to a nucleic acid sequence refers to a nucleic acid sequence that differs from the referenced sequence, but that encodes a polypeptide having the same amino acid sequence as that encoded by the referenced sequence.

Additionally, it will be appreciated by persons skilled in the art that partial sequences often work as effectively as full-length versions. The ways in which the nucleotide sequence may be varied or shortened are well known to persons skilled in the art, as are ways of testing the suitability or effectiveness of the altered genes. In certain embodiments, suitability and/or effectiveness of the altered gene may easily be tested by, for example, conventional gas chromatography. All such variations of the genes are therefore included as part of the present disclosure.

The term “isolated” and its grammatical equivalents as used herein refer to the removal of a nucleic acid from its natural environment. The term “purified” and its grammatical equivalents as used herein refer to a molecule or composition, whether removed from nature (including genomic DNA and mRNA) or synthesized (including cDNA) and/or amplified under laboratory conditions, that has been increased in purity, wherein “purity” is a relative term, not “absolute purity.” It is to be understood, however, that nucleic acids and proteins may be formulated with diluents or adjuvants and still for practical purposes be isolated. For example, nucleic acids typically are mixed with an acceptable carrier or diluent when used for introduction into cells. The term “substantially purified” and its grammatical equivalents as used herein refer to a nucleic acid sequence, polypeptide, protein or other compound which is essentially free, i.e., is more than about 50% free of, more than about 70% free of, more than about 90% free of, the polynucleotides, proteins, polypeptides and other molecules that the nucleic acid, polypeptide, protein or other compound is naturally associated with.

An “expression vector” or “vector” is any genetic element, e.g., a plasmid, a mini-circle, a nanoplasmid, chromosome, virus, transposon, behaving either as an autonomous unit of polynucleotide replication within a cell. (i.e. capable of replication under its own control) or being rendered capable of replication by insertion into a host cell chromosome, having attached to it another polynucleotide segment, so as to bring about the replication and/or expression of the attached segment. Suitable vectors include, but are not limited to, plasmids, transposons, bacteriophages and cosmids. Vectors may contain polynucleotide sequences which are necessary to affect ligation or insertion of the vector into a desired host cell and to affect the expression of the attached segment. Such sequences differ depending on the host organism; they include promoter sequences to effect transcription, enhancer sequences to increase transcription, ribosomal binding site sequences and transcription and translation termination sequences. Alternatively, expression vectors may be capable of directly expressing nucleic acid sequence products encoded therein without ligation or integration of the vector into host cell DNA sequences. In some embodiments, the vector is an “episomal expression vector” or “episome,” which is able to replicate in a host cell, and persists as an extrachromosomal segment of DNA within the host cell in the presence of appropriate selective pressure (see, e.g., Conese et al, Gene Therapy, 11:1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, Calif.) and pBK-CMV from Stratagene (La Jolla, Calif.) represent non-limiting examples of an episomal vector that uses T-antigen and the SV40 origin of replication in lieu of EBNA1 and oriP. Vector also may comprise a selectable marker gene.

The term “selectable marker gene” as used herein refers to a nucleic acid sequence that allows cells expressing the nucleic acid sequence to be specifically selected for or against, in the presence of a corresponding selective agent. Suitable selectable marker genes are known in the art and described in, e.g., International Patent Application Publications WO 1992/08796 and WO 1994/28143; Wigler et al, Proc. Natl. Acad. Sci. USA, 77: 3567 (1980); O'Hare et al, Proc. Natl. Acad. Sci. USA, 78: 1527 (1981), Mulligan & Berg, Proc. Natl. Acad. Sci. USA, 78: 2072 (1981); Colberre-Garapin et al., J. Mol. Biol, 150:1 (1981); Santerre et al., Gene, 30: 147 (1984): Kent et al., Science, 237: 901-903 (1987); Wigler et al Cell, 11: 223 (1977); Szybalska & Szybalski, Proc. Natl. Acad. Sci. USA, 48 2026 (1962); Lowy et al., Cell, 22: 817 (1980); and U.S. Pat. Nos. 5,122,464 and 5,770,359.

The term “coding sequence” as used herein refers to a segment of a polynucleotide that encodes for protein or polypeptide. The region or sequence is bounded nearer the 5′ end by a start codon and nearer the 3′ end with a stop codon. Coding sequences may also be referred to as open reading frames.

The term “operably linked” as used herein refers to refers to the physical and/or functional linkage of a DNA segment to another DNA segment in such a way as to allow the segments to function in their intended manners. A DNA sequence encoding a gene product is operably linked to a regulatory sequence when it is linked to the regulatory sequence, such as, for example, promoters, enhancers and/or silencers, in a manner, which allows modulation of transcription of the DNA sequence, directly or indirectly. For example, a DNA sequence is operably linked to a promoter when it is ligated to the promoter downstream with respect to the transcription initiation site of the promoter, in the correct reading frame with respect to the transcription initiation site and allows transcription elongation to proceed through the DNA sequence. An enhancer or silencer is operably linked to a DNA sequence coding for a gene product when it is ligated to the DNA sequence in such a manner as to increase or decrease, respectively, the transcription of the DNA sequence. Enhancers and silencers may be located upstream, downstream or embedded within the coding regions of the DNA sequence. A DNA for a signal sequence is operably linked to DNA coding for a polypeptide if the signal sequence is expressed as a pre-protein that participates in the secretion of the polypeptide. Linkage of DNA sequences to regulatory sequences is typically accomplished by ligation at suitable restriction sites or via adapters or linkers inserted in the sequence using restriction endonucleases known to one of skill in the art.

The term “induce” “induction” and its grammatical equivalents as used herein refer to an increase in nucleic acid sequence transcription, promoter activity and/or expression brought about by a transcriptional regulator, relative to some basal level of transcription.

The term “transcriptional regulator” refers to a biochemical element that acts to prevent or inhibit the transcription of a promoter-driven DNA sequence under certain environmental conditions (e.g., a repressor or nuclear inhibitory protein), or to permit or stimulate the transcription of the promoter-driven DNA sequence under certain environmental conditions (e.g., an inducer or an enhancer).

The term “enhancer” as used herein, refers to a DNA sequence that increases transcription of, for example, a nucleic acid sequence to which it is operably linked. Enhancers may be located many kilobases away from the coding region of the nucleic acid sequence or close to the coding region, and may mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers may be located upstream, within, or downstream of coding sequences.

The term “promoter” refers to a region of a polynucleotide that initiates transcription of a coding sequence. Promoters are located near the transcription start sites of genes, on the same strand and upstream on the DNA (towards the 5′ region of the sense strand). Some promoters are constitutive as they are active in all circumstances in the cell, while others are regulated becoming active in response to specific stimuli, e.g., an inducible promoter. The term “promoter activity” and its grammatical equivalents as used herein refer to the extent of expression of nucleotide sequence that is operably linked to the promoter whose activity is being measured. Promoter activity may be measured directly by determining the amount of RNA transcript produced, for example by Northern blot analysis or indirectly by determining the amount of product coded for by the linked nucleic acid sequence, such as a reporter nucleic acid sequence linked to the promoter.

“Inducible promoter” as used herein refers to a promoter which is induced into activity by the presence or absence of transcriptional regulators, e.g., biotic or abiotic factors. Inducible promoters are useful because the expression of genes operably linked to them may be turned on or off at certain stages of development of an organism or in a particular tissue. Non-limiting examples of inducible promoters include alcohol-regulated promoters, tetracycline-regulated promoters, steroid-regulated promoters, metal-regulated promoters, pathogenesis-regulated promoters, temperature-regulated promoters and light-regulated promoters. The inducible promoter may be part of a gene switch or genetic switch.

The term “termination sequence” as used herein, refers to a DNA sequence that ends transcription of, for example, a coding sequence. Termination sequences may be found after the coding regions and toward the 3′ region of the sense strand.

As used herein, the phrase “functional fragment” when used with reference to a polypeptide refers to a fragment of such polypeptide that possesses the primary function of the referenced polypeptide. For example, a functional fragment of a polypeptide that serves as a transmembrane domain is a fragment of that polypeptide that also serves as a transmembrane domain. When used with reference to a nucleic acid, the phrase “functional fragment” refers to a fragment that encodes a polypeptide having the same primary function as the polypeptide encoded by the referenced nucleic acid.

As used herein, the phrase “functional variant” when used with reference to a polypeptide refers to a polypeptide that is at least 80%, 85%, 90%, 95%, 96%, 97%, 98%, 99% or 100% identical to a reference polypeptide, or a fragment thereof, and possesses the primary function of the referenced polypeptide. For example, a functional variant of a polypeptide that serves as a transmembrane domain may be at least 80% identical to the reference polypeptide that also serves as a transmembrane domain. When used with reference to a nucleic acid, the phrase “functional variant” refers to a nucleic acid may differ from the referenced nucleic acid but encodes a polypeptide having the same primary function as the polypeptide encoded by the referenced nucleic acid.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Pinciples of Protein Structure., Springer-Verlag, New York (1979)). According to such analyses, groups of arnino acids may be defined where amino acids within a group exchange preferentially with each other, and therefore resemble each other most in their impact on the overall protein structure (Schulz, G. E. and Schirmer, R. H., supra). Examples of conservative mutations include amino acid substitutions of amino acids within the sub-groups above, for example, lysine for arginine and vice versa such that a positive charge may be maintained; glutamic acid for aspartic acid and vice versa such that a negative charge may be maintained; serine for threonine such that a free —OH may be maintained; and glutamine for asparagine such that a free —NH2 may be maintained.

In some embodiments, “functional variants” may also comprise the amino acid sequence of the reference protein with at least one non-conservative amino acid substitution. The term “non-conservative mutations” involve amino acid substitutions between different groups, for example, lysine for tryptophan, or phenylalanine for serine, etc. In this case, it is preferable for the non-conservative amino acid substitution to not interfere with, or inhibit the biological activity of, the functional variant. The non-conservative amino acid substitution may enhance the biological activity of the functional variant, such that the biological activity of the functional variant is increased as compared to the homologous parent protein. Amino acid substitutability is discussed in more detail, for example, in L. Y. Yampolsky and A. Stoltzfus, “The Exchangeability of Amino acids in Proteins,” Genetics 2005 August; 170(4):1459-1472, which is hereby incorporated by reference.

“Patient” or “subject” as used herein refers to a mammalian subject diagnosed with or suspected of having or developing cancer. In some embodiments, the term “patient” refers to a mammalian subject with a higher than average likelihood of developing cancer. Exemplary patients may be humans, apes, dogs, pigs, cattle, cats, horses, goats, sheep, rodents and other mammalians that may benefit from the therapies disclosed herein. Exemplary human patients may be male and/or female. “Patient in need thereof” or “subject in need thereof” is referred to herein as a patient diagnosed with or suspected of having a disease or disorder, for instance, but not restricted to cancer.

“Administering” is referred to herein as providing one or more compositions described herein to a patient or a subject. By way of example and not limitation, composition administration, e.g., injection, may be performed by intravenous (i.v.) injection, sub-cutaneous (s.c.) injection, intradermal (i.d.) injection, intraperitoneal (i.p.) injection, or intramuscular (i.m.) injection. One or more such routes may be employed. Parenteral administration may be, for example, by bolus injection or by gradual perfusion over time. Alternatively, or concurrently, administration may be by the oral route. Additionally, administration may also be by surgical deposition of a bolus or pellet of cells, or positioning of a medical device. In other examples, ocular administration is employed. In an embodiment, a composition of the present disclosure may comprise engineered cells or host cells expressing nucleic acid sequences described herein, or a vector comprising at least one nucleic acid sequence described herein, in an amount that is effective to treat or prevent inflammatory or autoimmune disease. A pharmaceutical composition may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

As used herein, the term “treatment”, “treating”, or its grammatical equivalents refers to obtaining a desired pharmacologic and/or physiologic effect. In some embodiments, the effect is therapeutic, i.e., the effect partially or completely cures a disease and/or adverse symptom attributable to the disease. In some embodiments, the term “treating” may include “preventing” a disease or a condition.

As used herein, a “treatment interval” refers to a treatment cycle, for example, a course of administration of a therapeutic agent that may be repeated, e.g., on a regular schedule. In some embodiments, a dosage regimen may have one or more periods of no administration of the therapeutic agent in between treatment intervals.

The terms “administered in combination” or “co-administration” or “co-administering” or “co-providing” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments may be partially additive, wholly additive, or greater than additive. The delivery may be such that an effect of the first treatment delivered is still detectable when the second is delivered.

In some embodiments, the first treatment and second treatment may be administered simultaneously (e.g., at the same time), in the same or in separate compositions, or sequentially. Sequential administration refers to administration of one treatment before (e.g., immediately before, less than 5, 10, 15, 30, 45, 60 minutes; 1, 2, 3, 4, 6, 8, 10, 12, 16, 20, 24, 48, 72, 96 or more hours; 4, 5, 6, 7, 8, 9 or more days; 1, 2, 3, 4, 5, 6, 7, 8 or more weeks before) administration of an additional, e.g., secondary, treatment. The order of administration of the first and secondary treatment may also be reversed.

As used herein, “treatment”, “treat”, and “treating” refer to reversing, alleviating, mitigating, or slowing the progression of, or inhibiting the progress of, a disorder or disease or symptoms associated with such disorder or disease, and as described in more detail herein.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 64, 6.5, 6.6, 6.7, 6.8, 6.9, and 7.0 are explicitly contemplated.

The power of unlimited self-renewal and the ability to differentiate into any cell type has long made pluripotent stem cells a keen area of interest for disease modeling. But with the difficulty and controversy associated with obtaining these stem cells, research has evolved to being able to generate pluripotent stem cells from somatic cells by reprogramming the somatic cell to allow transition to a less differentiated or undifferentiated state. The ability to derive these induced pluripotent stem cells (iPSCs) from a somatic cell type has proven to be a major advancement in the study of cancer and aging (Braganca et at, 2019). The transformation of somatic cells to induce pluripotency requires modification of the epigenetic and transcription factor framework from one cell state to another. This “reprogramming” by heterologous MUC1, MUC1-C or the MUC1 cytoplasmic domain, fragments, or functional variants thereof allows the induction of pluripotency in a somatic cell that begins the transition to a pluripotent state. As contemplated for the embodiments herein, induction of pluripotency is not achieved by modification of one or more heterologous Yamanaka factors, such as heterologous Oct3/4, Sox2, KI4, c-Myc, either individually or in any combination thereof, or modification of any regulatory sequences thereof.

Furthermore, the use of iPSCs as a potential immunotherapy is bolstered by the finding that iPSCs and cancer stem cells both share genetic and transcriptome signatures, including proteins markers for immune recognition. Without being bound by any theory, it is believed that this shared “expression signature” may allow the immune system to recognize an iPSC in lieu of a cancer stem cell, for the purposes of conferring immunity (Ouyang et al., 2019). Furthermore, using a patient's own cells to derive iPSCs as described herein for use as an immunotherapy against cancer offers the opportunity to develop personalize cancer vaccines specific to a patient.

Thus, the present disclosure relates to the engineering of a somatic cell to express MUC1, MUC1-C or the MUC1 cytoplasmic domain, fragments, or functional variants thereof, for inducing pluripotency to generate an iPSC, and use of the iPSC in a potential immunotherapy, for example a vaccine, that is personal to a patient. In some embodiments, the induction of pluripotency is not achieved by expressing one or more heterologous Yamanaka factors, such as heterologous Oct3/4, Sox2, Klf4, c-Myc, either individually or in any combination thereof, or expressing any heterologous regulatory sequences thereof.

Pluripotent cells are those that have the ability to differentiate into one of the three germ layers, MUC1, MUC1-C or the MUC1-C cytoplasmic domain, fragments, or functional variants thereof affect the pluripotent state of a cell and are regulators of self-renewal and differentiation for lineage specific differentiation and formation of germ layers. Thus, reprograming somatic cells to induce pluripotency includes the introduction of one or more pluripotency activators.

Thus, one embodiment described herein is a somatic cell engineered to introduce a heterologous nucleic acid sequence encoding MUC1, MUC1-C or the MUC1-C cytoplasmic domain, fragments, or functional variants thereof. Somatic cells may include any mammalian cell, for example, human (including from a patient suffering from one or more diseases) or animal cells, such as mice. These cells may be of any type or from any tissue, including but not limited to, skin, lung, intestine, heart, kidney, bladder tissue. The type of somatic cell useful in the present disclosure includes but is not limited to, endothelial cells, epithelial cells, epidermal cells, fibroblasts, keratinocytes, muscle cells, monocytes, immune cells, T cells, macrophages, hematopoietic cells. An adult stem cell capable of giving rise to all cell types is also considered a “somatic cell” as used herein, such as for example, neural stem cells. Thus, another embodiment described herein is an engineered somatic cell comprising a heterologous nucleic acid encoding MUC1, MUC1-C or the MUC1-C cytoplasmic domain, fragments, or functional variants thereof. Another embodiment described herein is a fibroblast comprising a heterologous nucleic acid encoding MUC1, MUC1-C or the MUC1-C cytoplasmic domain, fragments, or functional variants thereof. Another embodiment described herein is a fibroblast having at least one endogenous nucleic acid sequence encoding OCT3/4, SOX2, KLF4, and C-MYC, the fibroblast further comprising a heterologous nucleic acid sequence encoding MUC1-C operably linked to at least one regulatory sequence, wherein expression of MUC1-C is in an amount that induces pluripotency. Another embodiment described herein is a fibroblast having at least one nucleic acid sequence encoding OCT3/4, SOX2, KLF4, and C-MYC, wherein the at least one nucleic acid sequence encoding OCT3/4, SOX2, KLF4, and C-MYC consists of at least one endogenous nucleic acid sequence encoding OCT3/4, SOX2, KLF4, and C-MYC, the fibroblast further comprising a heterologous nucleic acid sequence encoding MUC1-C operably linked to at least one regulatory sequence, wherein expression of MUC1-C is in an amount that induces pluripotency.

In one aspect, the fibroblast comprises a dermal fibroblast, a lung fibroblast or an intestinal fibroblast. Fibroblasts from other tissues may also be used.

MUC1 appeared in mammals to protect epithelia from inflammation and damage induced by exposure to the external environment (Kufe, 2009, 2020). MUC1 encodes a protein that undergoes autoproteolytic cleavage into N-terminal (MUC1-N) and C-terminal (MUC1-C) subunits (Kufe, 2009, 2020). In response to loss of epithelial homeostasis, (i) MUC1-N is shed from the cell surface into the protective mucous barrier, and (ii) the transmembrane MUC1-C subunit activates proliferative and remodeling pathways associated with the process of wound healing (Kufe, 2020).

In one aspect, the heterologous MUC1 gene induces pluripotency. In another aspect, the heterologous MUC1 protein, fragments, or functional variants thereof, induces pluripotency. In another aspect, the heterologous MUC1-C subunit, fragments, or functional variants thereof, induces pluripotency. In yet another aspect, the heterologous MUC1-C extracellular domain, fragments, or functional variants thereof, induces pluripotency. In another aspect, the heterologous MUC1-C cytoplasmic domain, fragments, or functional variants thereof, induces pluripotency. In one aspect, one or more heterologous Yamanaka factors are not included in and, thus, do not induce pluripotency in the engineered somatic cell, such as a fibroblast. In another aspect, heterologous Oct3/4, Sox2, Klf4, c-Myc, either individually or in any combination thereof, are not included in and, thus, do not induce pluripotency in the engineered somatic cell, such as a fibroblast.

The wild-type MUC1-C coding sequence is 477 nucleotides in length and when expressed, is a transmembrane protein with a 58 amino acid long extracellular domain, a 28 amino acid long transmembrane domain and a 72 amino acid long cytoplasmic domain (Kufe, 2009). Without being bound by any theory, the cytoplasmic domain is believed to be involved in nuclear import and activation of various inflammatory pathways. (Kufe, 2009). SEQ ID NO: 1 describes the DNA sequence of MUC1-C and SEQ ID NO: 2 describes the amino acid sequence of MUC1-C with the extracellular domain italicized, the transmembrane domain underlined, and the cytoplasmic domain in lower caps. Target nucleic acid regions are shown in bold.

SEQ ID NO: 1: DNA sequence of MUC1-C
(SEQ ID NO: 1)
TCTGTGGTGGTACAATTGACTCTGGCCTTCCGAGAAGGTACCATCAATG
TCCACGACGTGGAGACACAGTTCAATCAGTATAAAACGGAAGCAGCCTC
TCGATATAACCTGACGATCTCAGACGTCAGCGTGAGTGATGTGCCATTT
CCTTTCTCTGCCCAGTCTGGGGCTGGGGTGCCAGGCTGGGGCATCGCGC
TGCTGGTGCTGGTCTGTGTTCTGGTTGCGCTGGCCATTGTCTATCTCAT
TGCCTTGGCTGTCtgtcagtgccgccgaaagaactacgggcagctggac
atctttccagcccgggatacctaccatcctatgagcgagtaccccacct
accacacccatgggcgctatgtgccccctagcagtaccgatcgtagccc
ctatgagaaggtttctgcaggtaatggtggcagcagcctctcttacaca
aacccagcagtggcagccacttctgccaacttgtag.
SEQ ID NO: 2: Amino acid sequence of MUC1-C
(SEQ ID NO: 2)
SVVVQLTLAFREGTINVHDVETQFNQYKTEAASRYNLTISDVSVSDVPF
PFSAQSGAGVPGWGIALLVLVCVLVALAIVYLIALAVcqcrrknygqld
ifpardtyhpmseyptyhthgryvppsstdrspyekvsagnggsslsyt
npavaatsanl*.

As disclosed herein, engineering somatic cells (e.g., fibroblasts) with a heterologous MUC1 gene and expression of MUC1 protein, fragment or functional variant thereof, including the MUC1-C or MUC1-C cytoplasmic domain coding sequences, induces pluripotency. In one aspect, the MUC1 nucleic acid sequence comprises SEQ ID NO: 1, or a functional variant thereof (e.g., a nucleic acid having at least about 80%, 85%, 90%, 95%, 96%, 97%, 98%, or 99% sequence identity with SEQ ID NO: 1), a codon degenerate variant of SEQ ID NO: 1. In another aspect, the MUC1 amino acid sequence comprises SEQ ID NO: 2 or a functional variant thereof (e.g., an amino acid having at least about 80%, 85%, 90%, 95%, 96%, 97%, 98%, or 99% sequence identity with SEQ ID NO: 2). In another aspect, the MUC1-C subunit comprises the wild-type sequence or a functional variant thereof (e.g., an amino acid having at least about 80%, 85%, 90%, 95%, 96%, 97%, 98%, or 99% sequence identity with the wild-type sequence). In another aspect, the MUC1-C cytoplasmic domain comprises the wild-type sequence or a functional variant thereof (e.g., an amino acid having at least about 80%, 85%, 90%, 95%, 96%, 97%, 98%, or 99% sequence identity with the wild-type sequence).

Although somatic cells may express low or undetectable levels of MUC1, MUC1-C subunit or MUC1-C cytoplasmic domain, their expression using exogenous nucleic acids may be required for conversion of the somatic cell to an iPSC. Thus, in one aspect is an oligonucleotide means for inducing pluripotency operably linked to at least one regulatory sequence. In one aspect, the oligonucleotide means for inducing pluripotency comprises the nucleic acid sequence of MUC1 or fragments thereof. In another aspect, the oligonucleotide means for inducing pluripotency comprises the nucleic acid sequence of MUC1-C or fragments thereof. In another aspect, the oligonucleotide means for inducing pluripotency comprises the nucleic acid sequence of MUC1-C extracellular domain or fragments thereof. In yet another aspect, the oligonucleotide means for inducing pluripotency comprises MUC1-C extracellular domain or fragments thereof. In one aspect, the oligonucleotide means for inducing pluripotency comprises the nucleic acid sequence of SEQ ID NO: 1 or a functional variant thereof (e.g., a nucleic acid having at least about 80%, 85%, 90%, 95%, 96%, 97%, 98%, or 99% sequence identity with SEQ ID NO: 1), or a codon degenerate variant of SEQ ID NO: 1. In another aspect, the nucleic acid means for inducing pluripotency comprises a nucleic acid encoding a polypeptide comprising the amino acid sequence of SEQ ID NO: 2 or a functional variant thereof (e.g., a nucleic acid having at least about 80%, 85%, 90%, 95%, 96%, 97%, 98%, or 99% sequence identity with SEQ ID NO: 2.

Accordingly, the somatic cells described herein may be engineered by any suitable method known in the art. For example, the engineered somatic cells described herein may be made by the introduction of a DNA construct into the somatic cell by viral vector transfection or transduction. DNA constructs may include all the necessary regulatory elements required to optimize expression of heterologous MUC1, MUC1-C or the MUC1 cytoplasmic domain, or fragment thereof, in the somatic cell, including promoters, termination sequences, enhancers and other regulatory elements known in the art. For example, cDNA may be inserted into an expression vector and operably linked to at least one regulatory sequence. The choice of expression vector is not limited to a specific vector and any suitable vector may be used. In one aspect, the promoter which drives expression from the cDNA expression construct is an inducible promoter. Regulatory sequences as used herein include promoters, enhancers, and termination sequences, and other expression control elements. Goeddel: Gene Expression Technology: Methods in Enzymology, Academia Press, San Diego, CA (1990). Design of the expression vector may depend on choice of host cell, type of protein to be expressed. Vector copy number expression control of markers may also be considered by the skilled artisan.

The present disclosure further contemplates a method of reprogramming a somatic cell to a less differentiated or undifferentiated state. Reprogramming of somatic cells results in an “induced pluripotent stem cell” (iPSC) of a lesser or de-differentiated state, than the somatic cell. The method comprises expressing heterologous MUC1, MUC1-C or the MUC1 cytoplasmic domain, or fragment thereof in a somatic cell, for regulating self-renewal and initiating transition of the differentiation state of the somatic cell to a less differentiated or de-differentiated state (pluripotent state). Thus, the resulting iPSCs may be partially differentiated or fully de-differentiated. In another aspect, is a method of reprogramming a somatic cell to an induced pluripotent stem cell comprising transforming a somatic cell with a means for inducing pluripotency and generating an induced pluripotent stem cell (iPSC).

Thus, another aspect of the present disclosure is a method of reprogramming a fibroblast to induce pluripotency comprising expressing heterologous MUC1, MUC1-C or the MUC1 cytoplasmic domain, or a fragment thereof in the fibroblast described herein; inducing pluripotency of the fibroblast; and generating an induced pluripotent stem cell from the fibroblast. Another embodiment described herein is a fibroblast comprising oligonucleotide means for inducing pluripotency operably linked to at least one regulatory sequence, wherein the means for inducing pluripotency comprises a heterologous nucleic acid sequence encoding MUC1, MUC1-C or the MUC1-C cytoplasmic domain, fragments or functional variants thereof, is expressed in an amount sufficient to induce pluripotency.

Expression may be in any amount effective to induce pluripotency. Expression levels may be measured by any means known in the art, including the use of Western blot, ELISA and qRT-PCT using RNA-seq datasets. In one aspect, the amount of expression of MUC1 or fragments thereof is in an amount effective to induce pluripotency. In another aspect, the amount of expression of MUC1-C subunit, fragments, or functional variants thereof is in an amount effective to induce pluripotency. In yet another aspect, the amount of expression of MUC1-C cytoplasmic domain, fragments, or functional variants thereof is in an amount effective to induce pluripotency.

Because the iPSCs are generated from a somatic cell, the iPSCs are autologous to the somatic cell or to the donor from which they came. Thus, for example, if a dermal fibroblast sample is obtained from a human patient, and subsequently the fibroblast is transfected to express the MUC1-C coding sequences and induce pluripotency, the resulting iPSC generated from the somatic cell, whether partially differentiated or fully undifferentiated, is autologous to the patient. Thus, in one aspect, the iPSC is autologous. As such, the resulting iPSC may be considered “personalized” to the patient from which the somatic cells were obtained.

Patient derived iPSCs provide an opportunity for the treatment of cancer because of certain shared characteristics with cancer cells. Like cancer cells, iPSCs are capable of unlimited self-renewal and share genetic and transcriptome signatures, including same or similar antigens for immune recognition. It is well known that cancer cells escape immune surveillance by a variety of mechanisms, thus the shared expression signature of iPSCs may provide an opportunity to induce immunity against the cancer itself. Furthermore, the autologous nature of the iPSC may allow for a personalized cancer therapy using patient derived iPSCs and reduce the possibility of immunogenicity.

Suppression of the antigen presentation machinery is a hurdle in cancer immunity (Charette et al, 2016). An anti-tumor immune response is the activation or priming of naive antigen-specific T cells and other immune cells by professional antigen-presenting cells (APCs), such as dendritic cells (DC). Dendritic cells capture, process and display peptide antigens complexed with major histocompatibility complex (MHC) to induce immunity. Dendritic cells may be found throughout tissue types and provide immune surveillance, collecting and processing of antigens for presentation.

Thus, without being bound by any theory, it is believed that since iPSCs are unable to efficiently present peptides complexed with MHC, fusing an iPSC to a dendritic cell will allow the dendritic cell to present the iPSC peptides. Because the expression signature of the iPSC is similar, or substantially similar, to the cancer cell or cancer stem cell, presentation of the iPSC peptides by the dendritic cell may thus illicit an immune response to the cancer cell or cancer stem cells, and induce immunity.

Thus, another aspect of the instant disclosure is a hybrid cell comprising a dendritic cell and the iPSC derived from a patient somatic cell as described in the instant disclosure. Like the iPSC, the dendritic cell may also be autologous to the patient. The hybrid cell may be the result of an iPSC cell fused to a dendritic cell to form, for example, a heterokaryon. Heterokaryons are a single cell with two separate nuclei formed by the experimental fusion of two genetically different cells. In another aspect, the iPSC expresses an antigen that is the same or substantially similar to that of a cancer cell or cancer stem cell. In another aspect, the hybrid cell presents the iPSC peptide antigen derived from the intracellular proteins expressed from the shared expression signature.

The iPSCs of the present disclosure may also be utilized to achieve the desired pharmacological or immunological effect by administration to a patient in need thereof in an appropriately formulated pharmaceutical composition. A patient, for the purpose of this disclosure, is a domestic animal or a human, in need of treatment for a particular condition or disease. Therefore, in one aspect of the present disclosure is a pharmaceutical composition comprising a therapeutically effective amount of the iPSCs derived from reprogramming of a somatic cell as described herein, and a pharmaceutically acceptable carrier. Another aspect is pharmaceutical composition comprising a therapeutically effective amount of the hybrid cell comprising iPSCs derived from reprogramming of a somatic cell as described herein, and a pharmaceutically acceptable carrier.

The term “composition” as used herein is intended to encompass a product comprising specific ingredients in specified amounts, as well as any product which results, directly or indirectly, from combination of the specified ingredients in the specified amounts. By “pharmaceutically acceptable” it is meant the carrier, diluent or excipient must be compatible with the other ingredients of the formulation, including the antisense oligonucleotide, and not deleterious to the recipient thereof. A “pharmaceutically acceptable carrier” is any carrier which is relatively non-toxic and innocuous to a patient at concentrations consistent with effective activity of the active ingredient so that any side effects ascribable to the carrier do not vitiate the beneficial effects of the active ingredient.

Appropriate pharmaceutical compositions comprising the iPSCs or hybrid cells comprising iPSCs, where the iPSC is derived from a somatic cell are contemplated herein, and are based partly on the specific tissues, and cell types involved. Pharmaceutical compositions appropriate for the cells of the instant disclosure may thus be formulated according to any means known in the art. (See for example: Remington's Pharmaceutical Sciences, 15th Edition, chapter 33; Gagliardi et al., 2021; or Hammond et al., 2021). In one aspect, the cells of the present disclosure may be formulated as a vaccine for administration to induce immunity. Vaccine formulations are known in the art.

The pharmaceutical compositions for the administration of the cells of this disclosure may conveniently be presented in unit dosage form and may be prepared by any of the methods well known in the art of pharmacy. All methods include the step of bringing the active ingredient into association with a carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition, the active object compound is included in an amount sufficient to produce the desired effect upon the process or condition of diseases.

The cells or compositions described herein may be administered with a pharmaceutically-acceptable carrier using any effective conventional dosage unit forms, including, for example, injectable, parenterally, or the like. In some aspects, the compositions is administered ocularly, intravenously, intramuscularly, intra-arterially, intratumorally, or subcutaneously.

The compositions may be formulated as vaccines. Vaccine formulations known in the art and may be administered by any means known in the art. Pharmaceutical injectable compositions may be administered parenterally, i.e. intravenously, subcutaneously, and intramuscularly. In some aspects, a pharmaceutical composition that is a vaccine may also be administered intranasally or to tissue in the oral cavity, such as by administration sublingually or to buccal tissue. The site of vaccination should allow for proper antigen presentation to the immune system. The number of vaccinations and priming conditions may be adjusted accordingly during treatment.

The present disclosure also contemplates various methods for using the cells or pharmaceutical compositions described herein.

Thus, one aspect described herein is a method of inducing an immune response in a subject in need thereof comprising administering to the subject an effective amount of the cells or pharmaceutical compositions described herein. In one aspect, an immune response may comprise, for example, induction of T cells, such as cytotoxic T lymphocytes, B cells, macrophages and other immune effectors, as well as an increase in the titer of anti-cancer neutralizing antibodies.

Another aspect is a method of treating cancer comprising administering an effective amount of pluripotent stem cells obtained by reprogramming somatic cells from the patient. In one aspect, the pluripotent stem cells are induced pluripotent stem cells (iPSCs). In another aspect, the pluripotent stem cells are undifferentiated. In another aspect, the pluripotent stem cells are partially differentiated. In yet another aspect, the pluripotent stem cells are autologous.

Another aspect is a method of treating cancer in a subject in need thereof comprising administering to the subject an effective amount of the cells or pharmaceutical composition described herein. In another aspect the method further comprises presenting the antigen expression signature.

Also described herein is a method of making a personalized cancer vaccine comprising obtaining a fibroblast from a patient; introducing in the fibroblast, an exogenous nucleic acid sequence encoding MUC1, MUC1-C or the MUC1 cytoplasmic domain, or fragments thereof, operably linked to at least one regulatory sequence; expressing MUC1, MUC1-C or the MUC1 cytoplasmic domain, or fragments thereof, in the fibroblast; inducing pluripotency of the fibroblast; generating an iPSC from the pluripotent fibroblast; fusing the iPSC with an dendritic cell to generate a hybrid cell; formulating the hybrid cell into a pharmaceutical composition.

The cells of the methods described herein may be partially differentiated or fully undifferentiated or de-differentiated. Because iPSCs strongly resemble the cancer cell or cancer stem cell, iPSCs may be irradiated to reduce the potential for them to become tumorigenic themselves. Thus, in another aspect of the method, the cells or pharmaceutical composition is irradiated prior to administering to the patient.

The cells and compositions of the method may be useful for immunizing against any cancer or treating any cancer. Exemplary cancers contemplated herein include, but are not limited to, solid tumor cancers, hematopoietic cancers, acute myeloid leukemia, multiple myeloma, breast cancer, prostate cancer, colorectal cancer and hepatic cancer. In another aspect, the cancer cell may comprise a cancer stem cell, a tumorigenic cancer cell, a hematopoietic cancer cell, leukemia cell, a myeloma cell, a breast cancer cell, a prostate cancer cell, a colorectal cancer cell or a hepatic cancer cell.

In another aspect, the subject is a human or a patient. In another aspect, the effective amount is any amount required to demonstrate a therapeutic effect. The therapeutically effective dosage of the cells or pharmaceutical compositions of the instant disclosure may readily be determined for treatment of each desired indication. For example, the cell dosage (range from about 1×10⁶ to about 1×10⁹) may be used for a vaccine and adjusted as needed. The amount of cells or composition to be administered in the treatment of one of these conditions may vary widely according to such considerations as the cell type and dosage unit employed, the mode of administration, the period of treatment, the age and sex of the patient treated, and the nature and extent of the condition treated.

The specific initial and continuing dosage regimen for each patient will vary according to the nature and severity of the condition as determined by the attending diagnostician, the age of the patient, the diet of the patient, time of administration, route of administration, rate of excretion of the drug, drug combinations, and the like. The desired mode of treatment and number of doses of the cells or pharmaceutical composition of the present disclosure may be ascertained by those skilled in the art using conventional treatment tests.

Another aspect described herein is a method of treating cancer comprising administering the pharmaceutical compositions described herein, wherein the composition may be administered in combination with one or more chemotherapeutic agents, targeted inhibitors, immune checkpoint inhibitors, cell therapies, monoclonal antibodies, oncolytic virus therapies, cancer vaccines, or immune system modulators, including but not limited to the full spectrum of compositions and compounds which are known to be active in killing and/or inhibiting the growth of cancer cells. (Ouyang et al., 2019)

Chemotherapeutic agents, may include, but are not limited to DNA-interactive agents, antimetabolites, tubulin interactive agents, anti-hormonals, anti-virals, ODC inhibitors and other cytotoxics such as hydroxy-urea. Any of these agents are suitable for use in the methods of the present invention, Chemotherapeutic agents further include, but not limited to cisplatin, carboplatin, camptothecin, indolizino, irinotecan, diflomotecan, exatecan, gimatecan, irinotecan, karenitecin, lurtorecan, rubitecan, silatecan, topotecan.

Antibodies may be polyclonal or monoclonal antibodies, humanized or human, that bind to an epitope on any of the cancers described herein. Any suitable antibody targeting the specific cancer contemplated herein may be used. In one aspect is a method of treating cancer comprising administering to a subject in need thereof, an effective amount of a pharmaceutical composition comprising any of the cells or pharmaceutical composition described herein in combination with one or more antibody. Exemplary antibodies for use with the cells or pharmaceutical composition described herein include rituximab, trastuzumab, ibritumomab, cetuximab, bevacizumab, panitumumab, ofatumumab, ipilimumab, brentuximab vedotin, pertuzumab, ado-trastuzumab emtansine, obinutuzumab, ramucirumab, pembrolizumab, blinatumomab, nivolumab, dinutuximab, daratumumab, necitumumab, elotuzumab, atezolizumab, olaratumab, avelumab, durvalumab, inotuzumab ozogamicin, tisagenlecleucel, gemtuzumab ozogamicin, axicabtagene ciloleucel, mogamulizumab-kpkc, moxetumomab pasudotox-tdfk, cemiplimab-rwlc, polatuzumab vedotin-piiq, enfortumab vedotin-ejfv, or fam-trastuzumab. Other antibodies not described herein may also be used in combination with the cells or pharmaceutical compositions described herein.

Targeted inhibitors comprise any targeted therapy, including but not limited to, therapies that target a specific gene or protein. These may include targeted therapies specific to a type of cancer. Examples of targeted inhibitors include inhibitors of HER2, BCR-ABL, EGFR, and VEGF, PARP or kinase inhibitors.

In another aspect of the present disclosure the cells or pharmaceutical composition may be administered in combination with one or more additional therapeutic agent. Potential other drugs include but not limited to: chemotherapeutic drugs including but not limited to camptothecin, indolizino, irinotecan, diflomotecan, exatecan, gimatecan, irinotecan, karenitecin, lurtorecan, rubitecan, silatecan, topotecan; targeted inhibitors; and antibodies.

Depending on the individual medicaments utilized in a combination therapy for simultaneous administration, they may be formulated in combination (where a stable formulation may be prepared and where desired dosage regimes are compatible) or the medicaments may be formulated separately (for concomitant or separate administration through the same or alternative routes).

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those skilled in the art will readily recognize a variety of noncritical parameters that could be changed or modified to yield essentially similar results.

### EXAMPLES

Transfection of Human Dermal Fibroblast Cells with MUC-1C

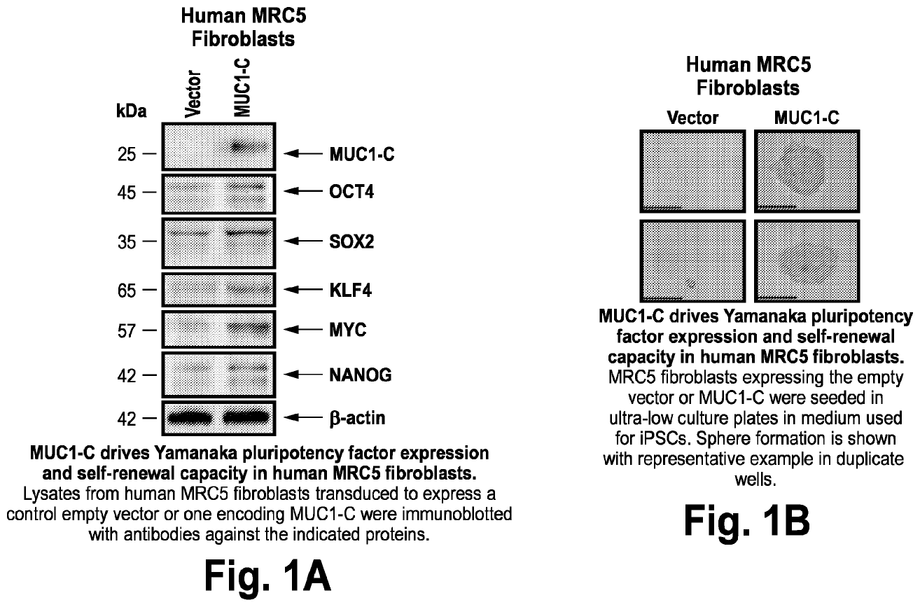

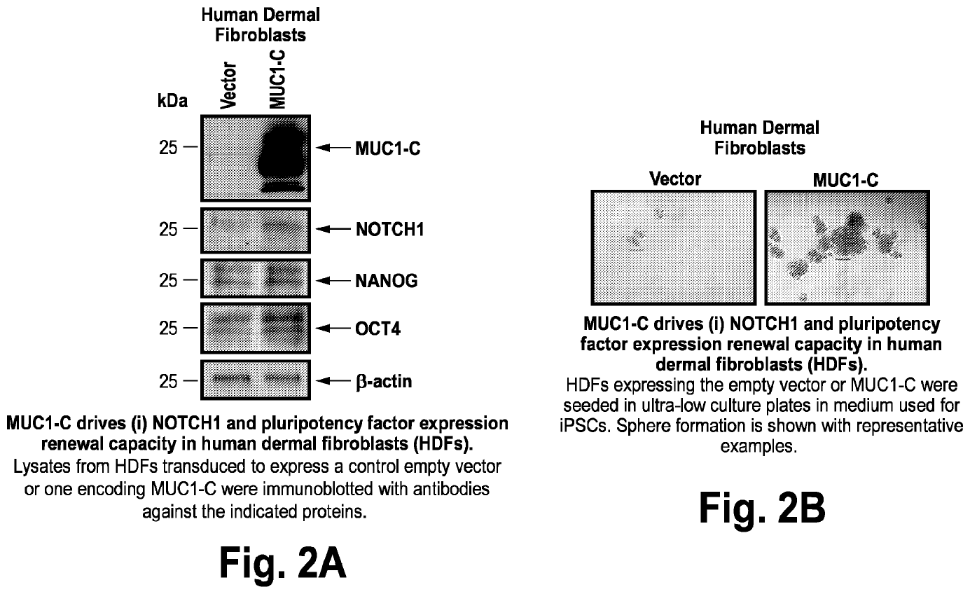

MUC1-C induces Yamanaka pluripotency factors, in association with self-renewal capacity, in the reprogramming of allogeneic human MRC5 fibroblasts. (FIGS. 2A and B), which have been used world-wide for decades as a platform for vaccine production. FIG. 1A. shows example lysates from human MRC5 fibroblasts transduced to express a control empty vector or one encoding MUC1-C that were immunoblotted with antibodies against the indicated proteins. FIG. 1B. shows example MRC5 fibroblasts expressing the empty vector or MUC1-C seeded in ultra-low culture plates in medium used for iPSCs. Sphere formation is shown with representative examples in duplicate wells.

Human dermal fibroblasts (HDFs) express low to undetectable MUC1-C levels. HDF's were prepared from small skin punch biopsies and transfected with an exogenous vector comprising the MUC1-C gene by methods commonly used and known in the art. Lysates from the HDFs transduced to express a control empty vector or one encoding MUC1-C were then immunoblotted with antibodies against the relevant proteins (FIG. 2A). HDFs expressing the empty vector of MUC1-C were seeded in ultra-low culture plates in medium used for iPSCs. Sphere formation was observed (FIG. 2B). Transfection resulted in increased expression of MUC1-C and increased expression of various pluripotency factors including NANOG, OCT4 and NOTCH 1.

### RNA-Seq Analysis

Using RNA-seq analysis, the present study was performed to determine the impact of overexpression of MUC1-C on specific genetic pathways and whether they resemble those in cancer cells.

Total RNA from cells cultured in triplicates was isolated using Trizol reagent (Invitrogen). TruSeq Stranded mRNA (Illumina) was used for library preparation.

Raw sequencing reads were aligned to the human genome (GRCh38.74) using STAR. Raw feature counts were normalized and differential expression analysis using DESeq2. Differential expression rank order was utilized for subsequent gene set enrichment analysis (GSEA), performed using the fgsea (v1.8.0) package in R. Gene sets queried included those available through the Molecular Signatures Database (MSigDB).

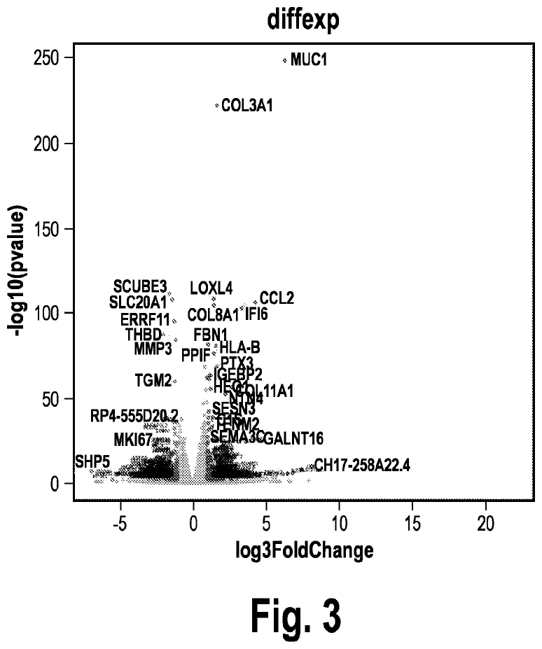

The data in FIGS. 3-12 demonstrate that overexpression of MUC1-C results in upregulation of genes associated with inflammatory pathways, and down regulation of genes associated with cell cycle regulation, similar to cancer cells.

FIG. 3 shows a volcano plot generated from RNA-seq analysis of HDFs without and with MUC1-C expression in which MUC1-C down-regulated genes are depicted on the left and up-regulated genes on the right.

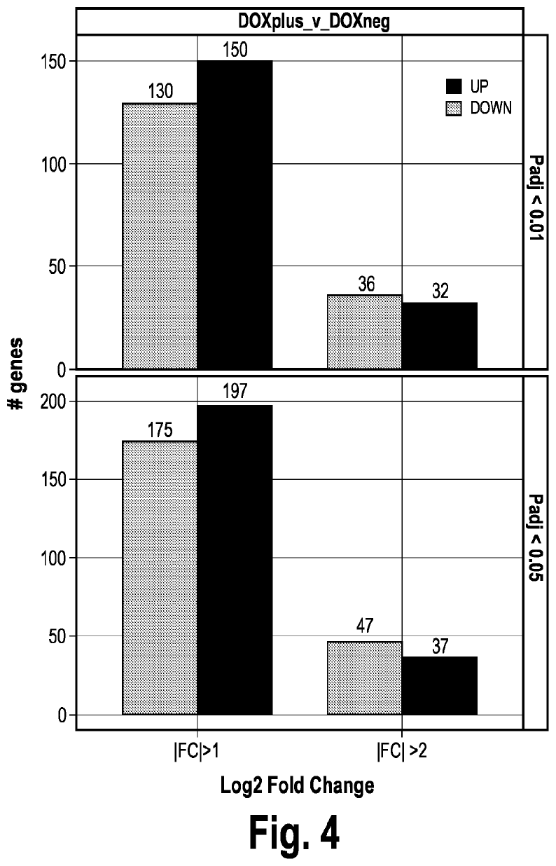

FIG. 4 shows numbers of MUC1-C-induced down-regulated and up-regulated genes using the indicated Log2 Fold Change cutoffs.

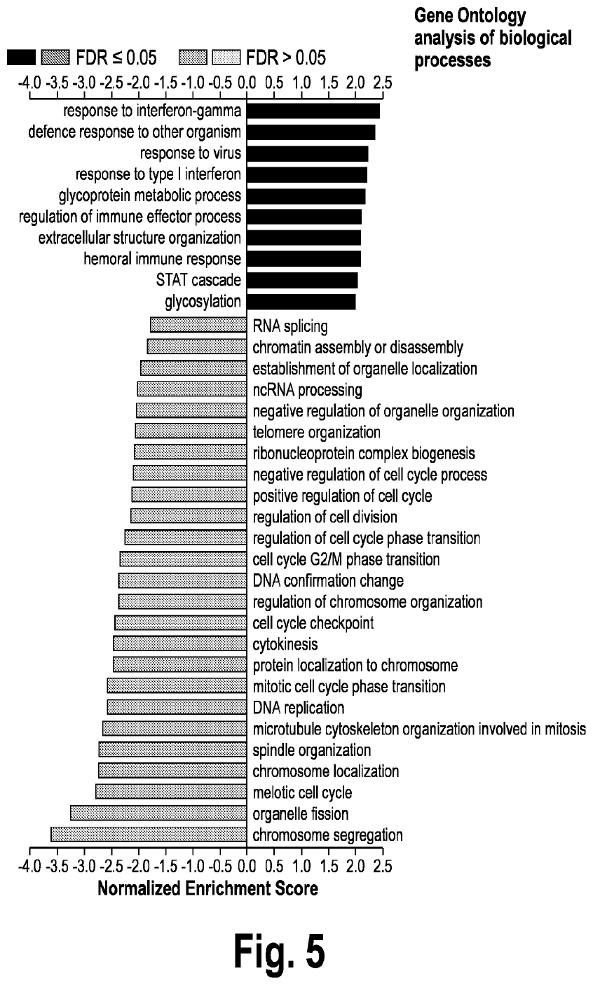

FIG. 5 shows analysis of the RNA-seq data using a Gene Ontology gene signature set, which demonstrates that MUC1-C (i) up-regulates the interferon type 1/II responses and other inflammatory associated pathways, and (ii) down-regulates specific pathways associated with cell cycle progression.

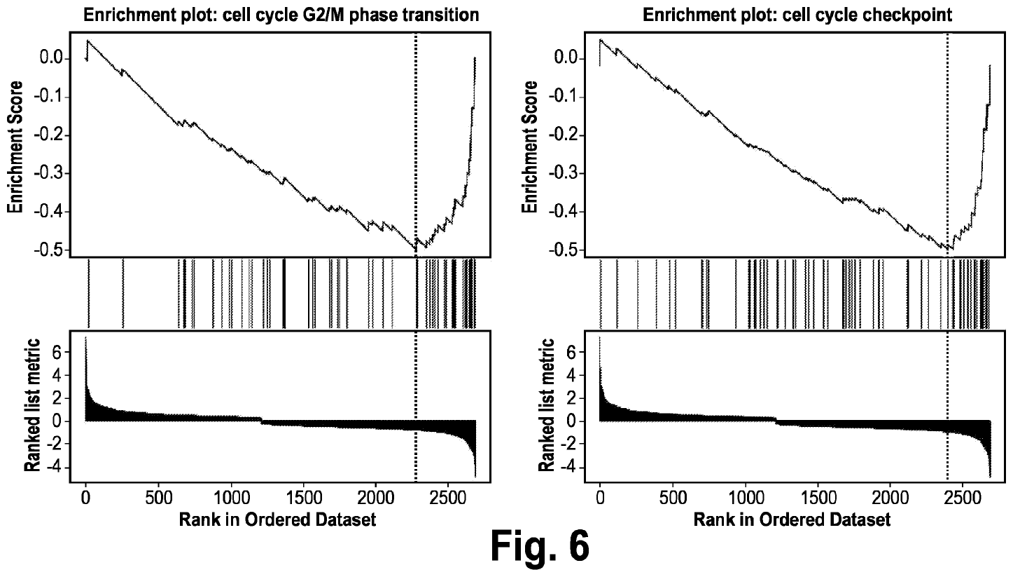

FIG. 6 shows Gene Set Enrichment Analysis (GSEA) of the RNA-seq data using the cell cycle G2/M phase and cell cycle checkpoint gene signatures down regulated by MUC1-C expression.

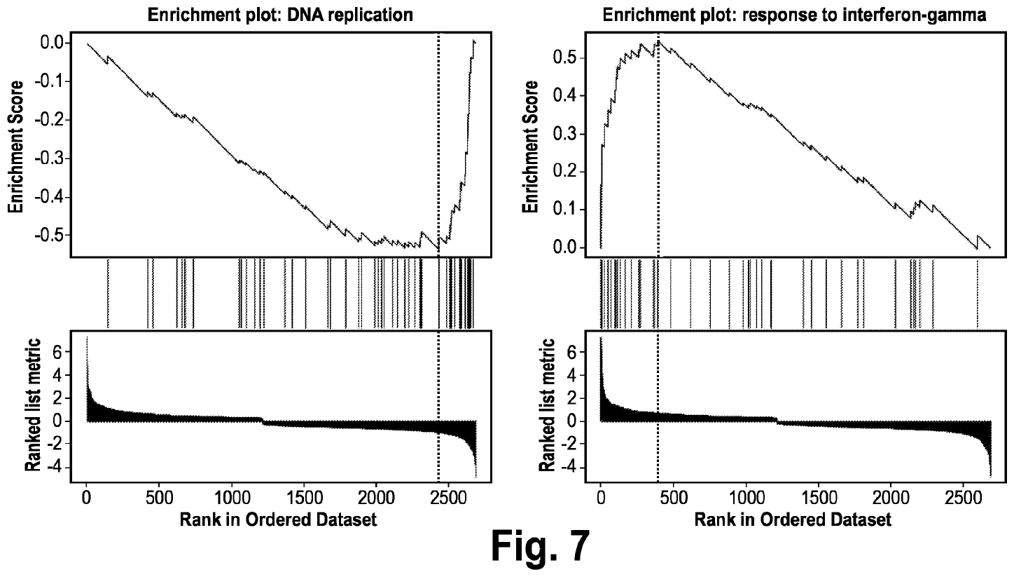

FIG. 7 shows GSEA of the RNA-seq data using the DNA regulation and response to interferon gamma gene signatures.

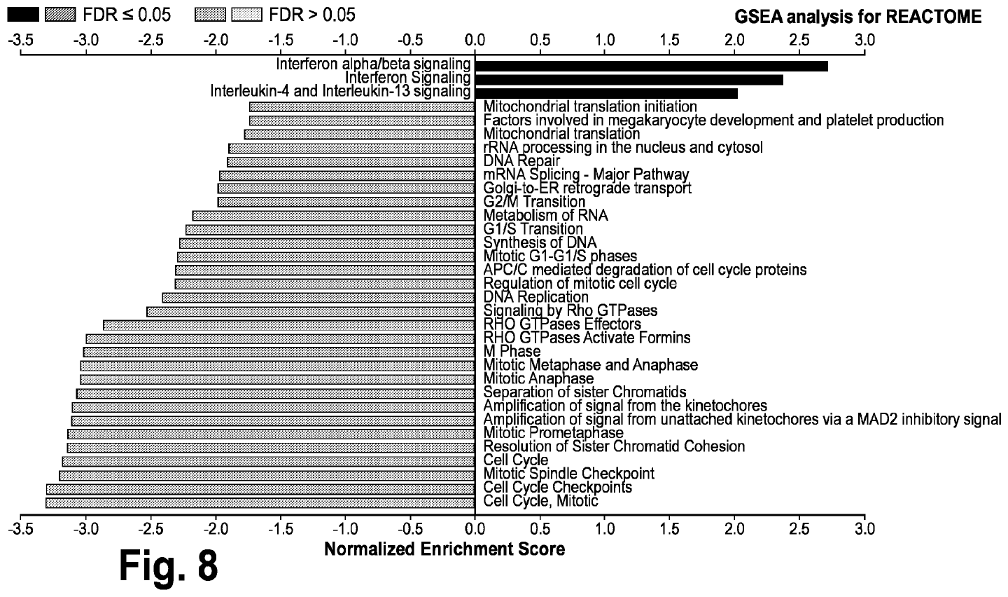

FIG. 8 shows GSEA of the RNA-seq data using the indicated Reactome gene signatures.

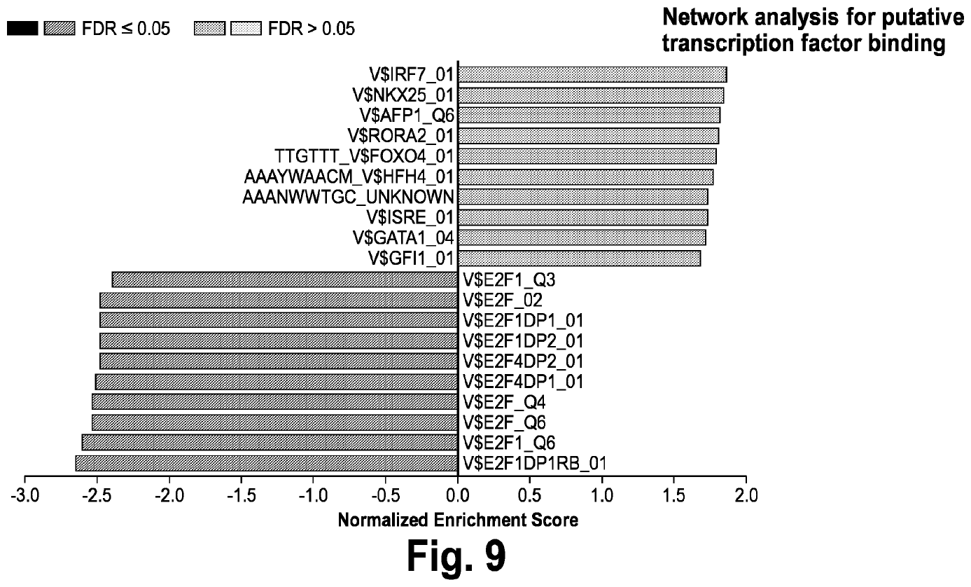

FIG. 9 shows network analysis of the RNA-seq data for putative transcription factor binding, which indicates that MUC1-C up-regulates transcription factors associated with inflammatory pathways and down-regulates those associated with cell cycle progression.

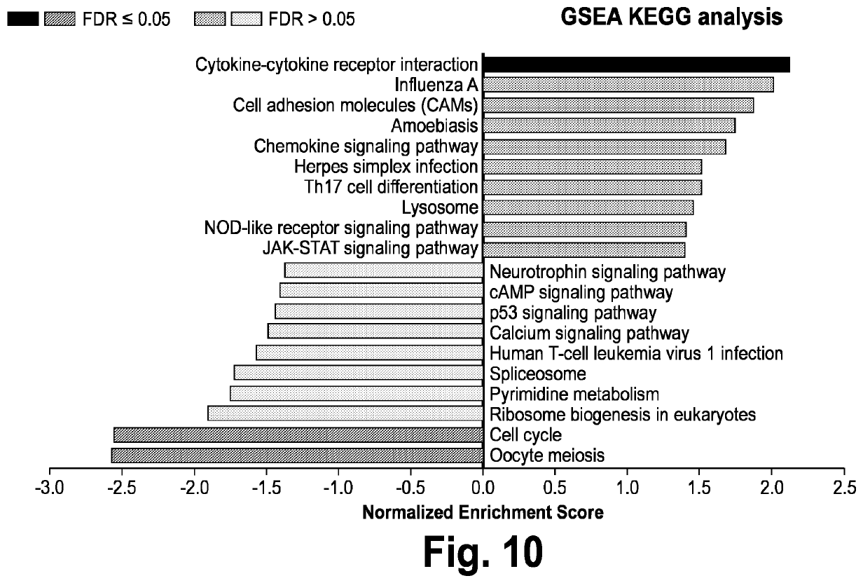

FIG. 10 shows GSEA analysis of the RNA-seq data using the indicated KEGG pathways

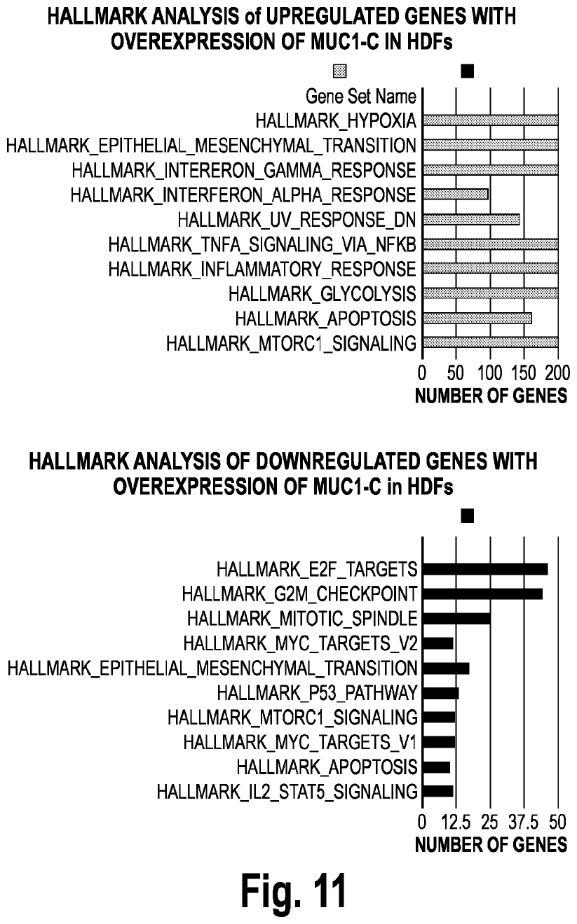

FIG. 11 shows GSEA of the RNA-seq data using the indicated HALLMARK genes signatures that are up-regulated (left) and down-regulated (right) by MUC1-C expression.

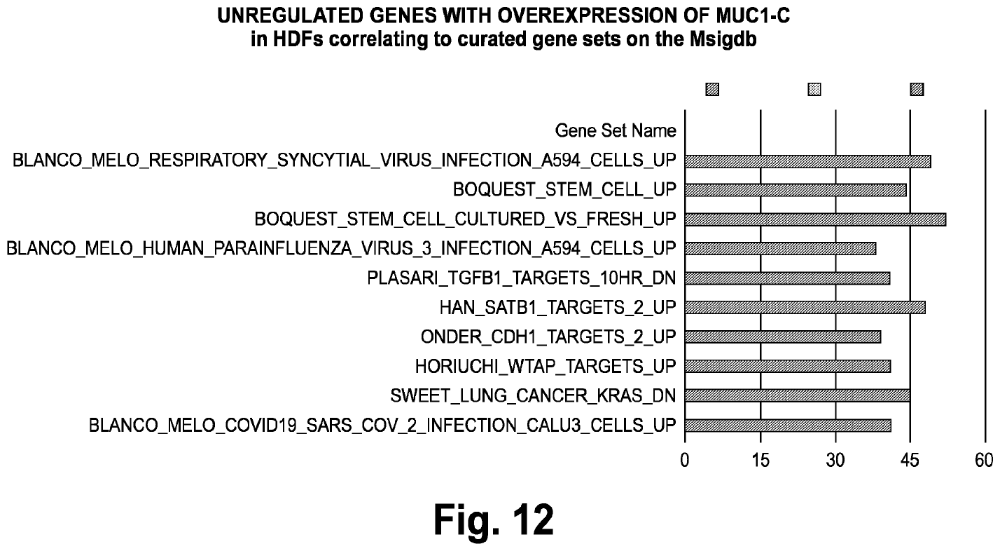

FIG. 12 shows GSEA of the RNA-seq data using the other indicated curated gene sets in the Molecular Signature Database (MSigDB).

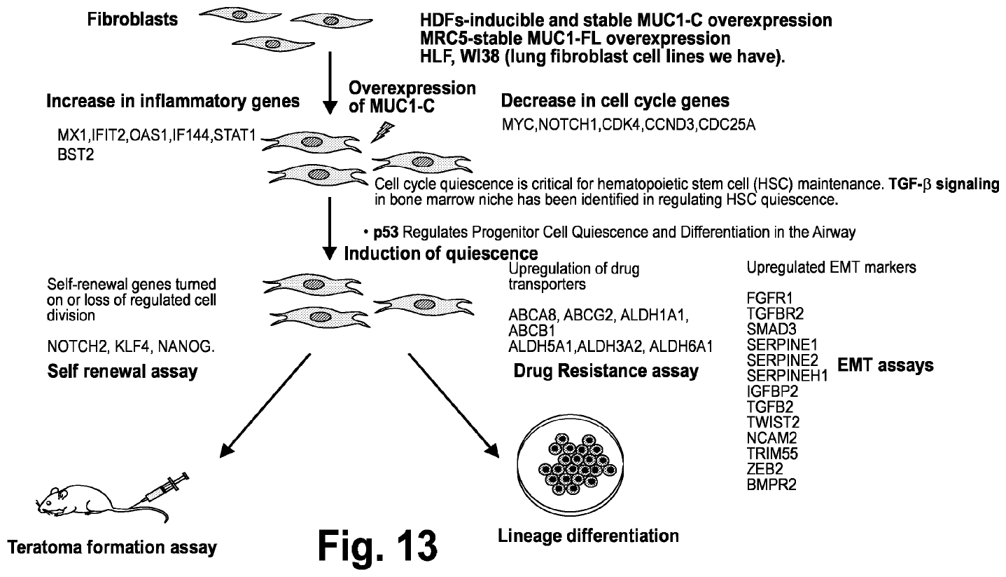

FIG. 13 describes the result of overexpression of MUC1-C in human dermal fibroblasts, resulting in upregulation of inflammatory genes and downregulation of cell cycle genes, the induction of quiescence, and the resulting impact on self-renewal or cell cycle regulation, upregulation of drug transporters and upregulation of EMT markers. Further studies may include performing a teratoma formation assay on immunodeficient mice, drug resistance assays, lineage differentiation analysis and EMT assays.

Thus, the data demonstrates that MUC1-C activates similar gene signatures in fibroblasts in driving iPSCs that also acquire self-renewal capacity.

In this way, MUC1-C converts a fibroblast to an iPSC with similar gene signatures and characteristics of CSCs. Thus, MUC1-C is sufficient to accomplish regulation of the pluripotency in an efficient manner.

### Prophetic Examples

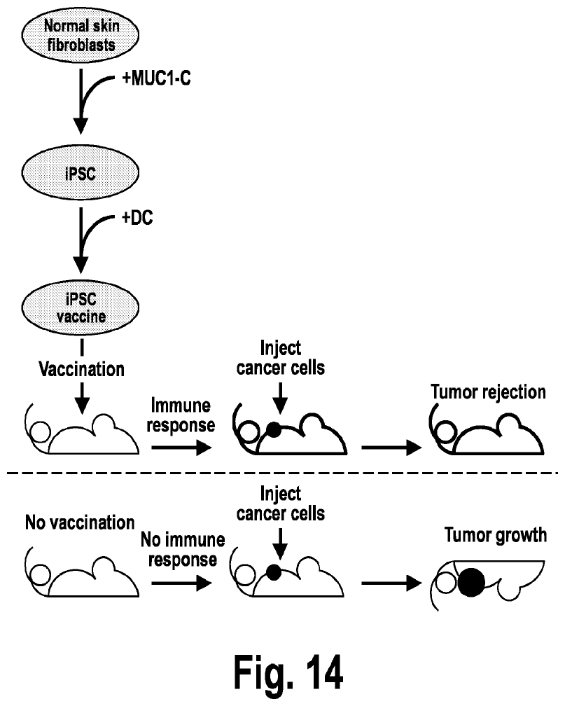

The RNA-seq data now provides the basis for identifying specific targets for a cancer vaccine. Future experiments will thus focus on fusing the iPSCs described herein with autologous dendritic cells to achieve fusion and subsequent presentation of the expression signature gene products as peptides to the immune system, to generate the iPSC-DC vaccine described herein (See, FIG. 14 and Avigan et al., Dendritic/Tumor Fusion Cells as Cancer Vaccines, Seminars in Oncology, Vol. 39, Issue 3, (2012), pgs. 287-295, with regard to dendritic based fusion cells as cancer vaccines.) Thus, mice will be vaccinated with the vaccine and subsequently exposed to tumor cells to determine if immunity will be conferred.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The examples described herein are provided by way of illustration only and not by way of limitation. Those skilled in the art will readily recognize a variety of parameters that could be changed or modified to yield essentially similar results.

1. A fibroblast comprising a heterologous nucleic acid sequence encoding MUC1-C operably linked to at least one regulatory sequence, wherein expression of MUC1-C is in an amount that induces pluripotency.

2. The fibroblast of claim 1, wherein the nucleic acid sequence encodes a polypeptide comprising the amino acid sequence of SEQ ID NO: 2 or a functional variant thereof.

3. The fibroblast of claim 1, wherein the MUC1-C comprises MUC1-C cytoplasmic domain.

4. The fibroblast of claim 1, wherein the fibroblast is autologous to a patient.

5. The fibroblast of claim 1, wherein the fibroblast comprises a dermal fibroblast, a lung fibroblast, or an intestinal fibroblast.

6. The fibroblast of claim 1, further comprising a nucleic acid sequence encoding for at least one of OCT3/4, SOX2, KLF4, and C-MYC, wherein the at least one of OCT3/4, SOX2, KLF4, and C-MYC consists of an endogenous nucleic acid sequence.

7. A fibroblast comprising a pluripotency activator consisting of one or more of MUC1, MUC1-C, or MUC1-C cytoplasmic domain.

8. The fibroblast of claim 1, wherein the pluripotency activator is not OCT3/4, SOX2, KLF4, and C-MYC or combinations thereof.

9. A fibroblast comprising oligonucleotide means for inducing pluripotency, operably linked to at least one regulatory sequence, wherein expression of the oligonucleotide means for inducing pluripotency is in an amount that induces pluripotency.

10. The fibroblast of claim 9, wherein the oligonucleotide means for inducing pluripotency comprises the nucleic acid sequence of SEQ ID NO: 1, or a functional variant thereof.

11. The fibroblast of claim 9, wherein the oligonucleotide means for inducing pluripotency encodes a polypeptide comprising the amino acid sequence of SEQ ID NO: 2, or a functional variant thereof.

12. method of reprogramming a fibroblast to induce pluripotency comprising:
(a) expressing MUC1-C in the fibroblast of claim 1;
(b) inducing pluripotency of the fibroblast; and
(c) generating an induced pluripotent stem cell from the fibroblast.

13. The method of claim 12, wherein the induced pluripotent stem cell is undifferentiated.

14. The method of claim 12, wherein the induced pluripotent cell is autologous to the fibroblast.

15. A hybrid cell comprising:
(a) an autologous dendritic cell; and
(b) the induced pluripotent stem cell of claim 9;
wherein the dendritic cell and induced pluripotent stem cell are fused.

16. The hybrid cell of claim 15, wherein the induced pluripotent cell comprises an expression signature.

17. The hybrid cell of claim 16, wherein the expression signature comprises an expression signature substantially similar to the expression signatures of cancer cells or cancer stem cells.

18. The hybrid cell of claim 16, wherein the expression signature encodes proteins from which at least one peptide is derived.

19. A pharmaceutical composition comprising the hybrid cell of claim 1.

20. A method of inducing an immune response in a subject in need thereof comprising administering to the subject an effective amount of the pharmaceutical composition of claim 19.

21. A method of treating cancer in a subject in need thereof comprising administering to the subject an effective amount of the pharmaceutical composition of claim 19.

22. The method of claim 20, further comprising presenting the peptide.

23. The method of claim 22, further comprising inducing an immune response.

24. The method of claim 20, further comprising irradiating the hybrid cell prior to administration to the subject.

25. The method of claim 21, wherein the cancer comprises a solid tumor or liquid cancer.

26. A method of making a personalized cancer vaccine comprising:
(a) obtaining a fibroblast from a subject;
(b) introducing in the fibroblast, an exogenous nucleic acid sequence encoding MUC1-C operably linked to at least one regulatory sequence;
(c) expressing MUC1-C in the fibroblast;
(d) inducing pluripotency of the fibroblast;
(e) generating an autologous induced pluripotent stem cell from the fibroblast;
(f) fusing the autologous induced pluripotent stem cell with an autologous dendritic cell to generate a hybrid cell; and
(g) formulating the hybrid cell into a pharmaceutical composition.

27. The method of claim 26, wherein the induced pluripotent stem cell comprises an expression signature substantially similar to the expression signature of cancer cells or cancer stem cells.

28. The method of claim 26, wherein the expression signature encodes proteins from which at least one peptide is derived.

29. The method of claim 28, wherein the hybrid cell presents the peptide.
